(19) United States
(12) Patent Application Publication
(10) Pub. No.: US 2026/0108344 A1
(43) Pub. Date: Apr. 23, 2026

(54) BIODEGRADABLE STENTS

(71) Applicant: BOSTON SCIENTIFIC SCIMED, INC., Maple Grove, MN (US)

(72) Inventors: Rosangel Aimee Ramos Espinoza, Boston, MA (US); Colby Harris, Norfolk, MA (US); David Robert Wulfman, Minneapolis, MN (US); Justin Theodore Nelson, Vadnais Heights, MN (US); Danielle Frankson, Dayton, MN (US)

(73) Assignee: BOSTON SCIENTIFIC SCIMED, INC., Maple Grove, MN (US)

(21) Appl. No.: 19/357,828

(22) Filed: Oct. 14, 2025

Related U.S. Application Data
(60) Provisional application No. 63/707,579, filed on Oct. 15, 2024.

(51) Int. Cl. A61F 2/04; A61L 31/10; A61L 31/14

(52) U.S. Cl. CPC A61F 2/04; A61L 31/10; A61L 31/148; A61F 2002/041; A61F 2210/0004; A61F 2210/0076; A61F 2220/0008; A61F 2240/001; A61F 2250/0031; A61L 2400/18

(57) ABSTRACT

An example of a biodegradable biliary stent may comprise an elongated tubular member having a first end region, a second end region, and an intermediate region disposed therebetween, the elongated tubular member comprising at least one filament; and a biodegradable anchoring material that is coupled to and overlays a portion of the elongated tubular member, wherein the biodegradable anchoring material is coupled to and overlays at least to the first end region, the second end region, or both the first end region and the second end region.

### CROSS REFERENCE TO RELATED APPLICATIONS

The present application claims the benefit of U.S. Provisional Patent Application Ser. No. 63/707,579, filed Oct. 15, 2024, the disclosure of which is incorporated herein by reference.

### TECHNICAL FIELD

The present disclosure pertains to medical devices, and methods for manufacturing medical devices. More particularly, the present disclosure pertains to biodegradable biliary stents and methods of use and manufacture thereof.

### BACKGROUND

Implantable stents are devices that are placed in a body structure, such as a blood vessel or body cavity, to provide support and to maintain the structure open. These devices are manufactured by any one of a variety of different manufacturing methods and may be used according to any one of a variety of methods. Of the known medical devices, delivery systems, and methods, each has certain advantages and disadvantages. Biliary stents are medical devices used to maintain patency in the biliary system, which includes the bile ducts and gallbladder. These stents play a crucial role in managing various conditions related to the biliary and pancreatic systems, such as malignant biliary obstruction, pancreatic duct obstruction, biliary leaks, and post-ERCP pancreatitis. Biliary stents are designed to provide structural support and maintain duct patency for extended periods. The field of biliary stent technology encompasses a range of materials, designs, and deployment methods aimed at optimizing patient outcomes and minimizing complications associated with stent placement and removal. There is an ongoing need to provide alternative medical devices and delivery devices as well as alternative methods for manufacturing and using medical devices and delivery devices.

### BRIEF SUMMARY

This disclosure provides design, material, manufacturing method, and use alternatives for medical devices, namely stents. In a first example a biodegradable stent is provided. The biodegradable stent comprising: an elongated tubular member having a first end region, a second end region, and an intermediate region disposed therebetween, the elongated tubular member comprising at least one filament; and a biodegradable anchoring material that is coupled to and overlays a portion of the elongated tubular member, wherein the biodegradable anchoring material is coupled to and overlays at least to the first end region, the second end region, or both the first end region and the second end region.

Alternatively or additionally to any of the embodiments above, wherein the biodegradable anchoring material is coupled to and overlays both the first end region and the second end region of the elongated tubular member.

Alternatively or additionally to any of the embodiments above, wherein the biodegradable anchoring material is only coupled to and overlays the first end region, the second end region, or both the first end region and the second end region.

Alternatively or additionally to any of the embodiments above, wherein the biodegradable anchoring material is configured to degrade over time, allowing for removal of the biodegradable biliary stent after a predetermined indwell time.

Alternatively or additionally to any of the embodiments above, wherein the biodegradable anchoring material comprises least one of a magnesium-based alloy, one of poly-L-lactic acid (PLLA), polylactic acid (PLA), polyglycolic acid (PGA), polyglycolide-co-L-lactide acid (PGLA), polydioxanone (PDO), or polyglycolide-co-caprolactone (PGCL).

Alternatively or additionally to any of the embodiments above, biodegradable anchoring material defines a plurality of interstices.

Alternatively or additionally to any of the embodiments above, wherein the biodegradable anchoring material is a biodegradable lattice.

Alternatively or additionally to any of the embodiments above, wherein the elongated tubular member is a braided elongate tubular member, and wherein the biodegradable lattice is braided or woven into the first end region, the second end region, or both the first end region and the second end region.

Alternatively or additionally to any of the embodiments above, wherein the biodegradable anchoring material is a spun biodegradable matrix.

Alternatively or additionally to any of the embodiments above, wherein the spun biodegradable matrix further comprises an electrospun biodegradable matrix.

Alternatively or additionally to any of the embodiments above, wherein the biodegradable anchoring material includes an adhesive material.

Alternatively or additionally to any of the embodiments above, wherein the adhesive material comprises at least one of chitosan and transglutaminase.

Alternatively or additionally to any of the embodiments above, wherein the biodegradable anchoring material is functionalized, and wherein adhesive material is coupled to the functionalized biodegradable anchoring material.

Alternatively or additionally to any of the embodiments above, wherein the elongated tubular member is fully covered.

Alternatively or additionally to any of the embodiments above, wherein the elongated tubular member is a braided elongated tubular member, wherein the first end region is a flared first end region, and wherein the second end region is a flared second end region.

In another example a coated biodegradable stent is provided. The coated biodegradable stent comprising: a braided elongated tubular member having a first end region, a second end region, and an intermediate region disposed therebetween, the elongated tubular member comprising at least one filament and a coating overlaying at least a portion of the elongated tubular member, wherein the at least on filament is a non-biodegradable filament; and a biodegradable anchoring material coupled to a portion of the braided elongated tubular member, wherein the biodegradable anchoring material is coupled to at least to the first end, the second end, or both the first end region and the second end region of the braided elongated tubular member, wherein the biodegradable anchoring material comprises at least one of a magnesium-based alloy, polylactic acid (PLA), and polyglycolic acid (PGA) and is configured to provide anchoring and biodegrade over time.

In another example, a method of manufacturing a coated biodegradable stent is provided. The method comprising: providing a braided elongated tubular member having a first end region, a second end region, and an intermediate region disposed therebetween, the braided elongated tubular member comprising at least one filament; applying a coating to the braided elongated tubular member; and applying a biodegradable anchoring material to at least the first end region, the second end region, or both the first end region and the second end region of the braided elongated tubular member.

Alternatively or additionally to any of the embodiments above, wherein biodegradable anchoring material further comprises a biodegradable lattice, and wherein applying the biodegradable lattice comprises braiding or weaving the biodegradable lattice onto the first end region, the second end region, or both the first end region and the second end region.

Alternatively or additionally to any of the embodiments above, wherein biodegradable anchoring material further comprises a biodegradable anchoring coating, and wherein applying the biodegradable coating comprises electrospinning a biodegradable polymer onto at least the first end region, the second end region, or both the first end region and the second end region of the elongated tubular member while applying or cross-linking the coating to the braided elongated tubular member.

Alternatively or additionally to any of the embodiments above, further comprising forming an adhesion-enhanced biodegradable anchoring coating by: functionalizing the biodegradable anchoring coating by applying polyethylene glycol (PEG) or a PEG derivative to the biodegradable anchoring coating; and applying an adhesive material to the functionalized biodegradable coating to form the adhesion-enhanced biodegradable anchoring coating.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The description may be more completely understood in consideration of the following detailed description of various embodiments in connection with the accompanying drawings, in which:

FIG. 1 is a side view of an illustrative biodegradable stent;

FIG. 2A is an example of a biodegradable stent including a biodegradable anchoring material in the form of a biodegradable lattice;

FIG. 2B illustrates a section view of the biodegradable stent of FIG. 2A;

FIG. 2C illustrates a view of an end portion of the biodegradable stent of FIG. 2A;

FIG. 3A is an example of a biodegradable stent including a biodegradable anchoring material in the form of a spun biodegradable matrix;

FIG. 3B illustrates a section view of the biodegradable stent of FIG. 3A;

FIG. 3C illustrates a view of an end portion of the biodegradable stent of FIG. 3A; and

FIG. 4 is an illustrative method of forming a coated biodegradable stent.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or”unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

The technical field of this disclosure relates to biodegradable biliary stents, for instance, biodegradable biliary stents that are used in the management of various biliary and pancreatic conditions. Traditional approaches to biliary stenting have faced challenges related to stent migration, the need for repeat procedures for stent removal, and potential tissue damage during removal. These challenges have led to increased patient risks, higher healthcare costs, and reduced overall treatment efficacy.

The present disclosure addresses these challenges and other challenges by introducing a novel biodegradable biliary stent design that incorporates advanced coating technologies and materials. For instance, the biodegradable biliary stents herein include a biodegradable anchoring material (e.g., a biodegradable lattice or a spun biodegradable matrix) that provides initial anchoring capabilities within a lumen of a vessel, yet biodegrades over time to permit easy removal of the biodegradable biliary stents (e.g., after a predetermined indwell time period). That is, unlike some other stents which are not readily removable or repositionable, the biodegradable biliary stents herein are configured with a portion thereof that is configured to biodegrade (e.g., after a predetermined indwell time). In some embodiments, the biodegradable biliary stents herein also configured with a main stent body which is formed of a non-biodegradable material which allows the biodegradable biliary stents to retain structural integrity to ensure luminal patency is maintained over an operation lifetime of the biodegradable biliary stents (e.g., for a desired indwell time period), as compared to other approaches such as those which employ a main stent body that is formed of a biodegradable material. While the embodiments disclosed herein are discussed with reference to biliary stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, esophagus, urinary tract, prostate, brain, stomach and the like.

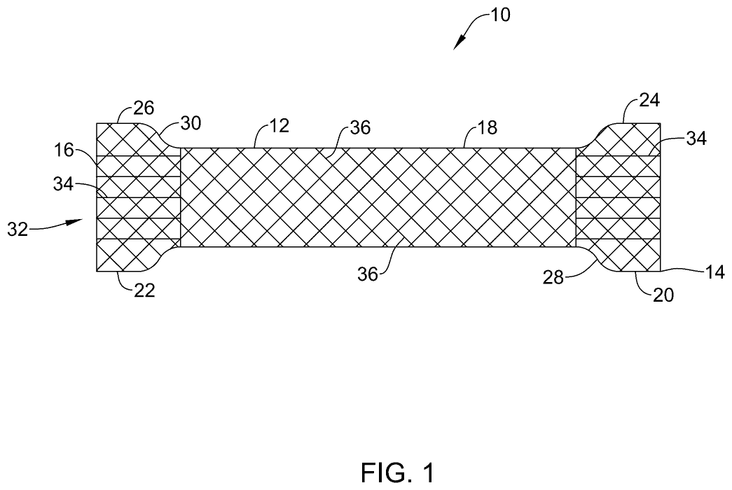

FIG. 1 illustrates a side view of an illustrative biodegradable stent 10, which in some instances may be a biodegradable biliary stent. In some instances, the stent 10 may be formed from an elongated tubular member 12. While the stent 10 is described as generally tubular, it is contemplated that the stent 10 may take any cross-sectional shape desired. The stent 10 may have a first, or proximal, end 14, a second, or distal, end 16, and an intermediate region 18 disposed between the first end 14 and the second end 16. The stent 10 may include a lumen 32 extending from a first opening adjacent to the first end 14 to a second opening adjacent to the second end 16 to allow for the passage of various material such as fluids, etc.

The stent 10 may be expandable from a first collapsed configuration (not explicitly shown) to a second expanded configuration. The stent 10 may be structured to extend across a stricture and to apply a radially outward pressure to the stricture in a lumen to open the lumen and allow for the passage of various materials such as fluids, etc.

The stent 10 may have a woven structure, fabricated from a number of filaments or struts 36. In some embodiments, the stent 10 may be braided with one filament. In other embodiments, the stent 10 may be braided with several filaments, as is found, for example, in the WallFlex®, WALLSTENT®, and Polyflex® stents, made and distributed by Boston Scientific, Corporation. In another embodiment, the stent 10 may be knitted, such as the Ultraflex™ stents made by Boston Scientific, Corporation. In still another embodiment, the stent 10 may be a laser cut tubular member, such as the EPIC™ stents made by Boston Scientific, Corporation. A laser cut tubular member may have an open and/or closed cell geometry including one or more interconnected filaments. In some instances, an inner and/or outer surface of the stent 10 may be entirely, substantially or partially, or fully covered with a polymeric covering or coating. For example, at least the outer surface of the stent may be fully covered such that the stent is manifested as a fully covered stent. The covering or coating (e.g., a silicone coating) which may help reduce tumor or tissue ingrowth.

In some instances, in the expanded configuration, the stent 10 may include a first end region 20 and a second end region 22. In some embodiments, the first end region 20 and the second end region 22 may include retention features or flared end regions (e.g., anti-migration flared regions) 24, 26 positioned adjacent to the first end 14 and the second end 16 of the stent 10. The anti-migration flared regions 24, 26 may be configured to engage an interior portion of the walls of a body lumen. In some embodiments, the retention features, or flared regions 24, 26 may have a larger diameter than an intermediate region 18 of the stent 10 to prevent the stent 10 from migrating once placed in the lumen of the body. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the anti-migration flared regions 24, 26. For example, the first end region 20 may include an anti-migration flare 24 while the second end region 22 may have an outer diameter similar to the intermediate region 18. It is further contemplated that the second end region 22 may include an anti-migration flare 26 while the first end region 20 may have an outer diameter similar to an outer diameter of the intermediate region 18. In some embodiments, the stent 10 may have a uniform outer diameter from the first end 14 to the second end 16. In some embodiments, the outer diameter of the intermediate region 18 may be in the range of 15 to 25 millimeters. The outer diameter of the anti-migration flares 24, 26 may be in the range of 20 to 30 millimeters. It is contemplated that the outer diameter of the stent 10 may be varied to suit the desired application.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease from a site in a lumen as well. For example, the stent 10 can be formed from alloys (e.g., non-biodegradable alloys) such as, but not limited to, nitinol and Elgiloy®. Depending the on material selected for construction, the stent 10 may be self-expanding or require an external force to expand the stent 10. In some embodiments, fibers may be used to make the stent 10, which may be cored fibers, for example, having an outer shell made of nitinol having a platinum core. It is further contemplated the stent 10 may be formed from polymers including, but not limited to, polyethylene terephthalate (PET).

The stent 10 may further include a biodegradable anchoring material 34 coupled to a portion of the elongated tubular member 12. The biodegradable anchoring material 34 can be attached at or overlay (e.g., a least partially cover or entirely cover) the first end region 20 and/or the second end region 22 of the stent 10. For instance, the biodegradable anchoring material 34 can be attached at and overlay at least a portion of the first end region 20 and/or the second end region 22 along an entire length (longitudinally) of first end region 20 and/or the second end region 22 of the stent. That is, the biodegradable anchoring material 34 can be coupled to and overlay an exterior (e.g., radial most) surface of a portion of the stent 10. For instance, the biodegradable anchoring material 34 can be attached to or overlay the first end region 20 and/or the second end region 22 of the stent 10, but is not attached to (e.g., does not overlay) the intermediate region 18 of the elongated tubular member 12, in some embodiments. Having the biodegradable anchoring material 34 be attached to or overlay the first end region 20 and/or the second end region 22 of the stent 10, but not be attached to (e.g., does not overlay) the intermediate region 18 the stent 10 can ensure that the stent 10 retains a desired mechanical performance (e.g., an ability of the stent 10 to contract during delivery and expand once delivered to a target site inside a body lumen).

For example, as illustrated in FIG. 1, the biodegradable anchoring material 34 can be attached to or overlay the first end region 20 and the second end region 22 of the stent 10, but is not attached to and does not overlay (e.g., does not overlay any portion of) the intermediate region 18 of the stent 10. In some embodiments, the biodegradable anchoring material 34 can be attached to or overlay the first end region 20 and/or the second end region 22 but not the intermediate region by initially coupling the biodegradable anchoring material 34 only to the first end region 20 and/or the second end region 22. However, in some embodiments the biodegradable anchoring material 34 can be attached to or overlay the first end region 20 and/or the second end region 22, but not the intermediate region 18 by application of the biodegradable anchoring material 34 to the entire elongated tubular member 12 and selectively removing (e.g., chemically removing and/or mechanically removing) a portion of the biodegradable anchoring material 34 from at least the intermediate region 18. The selective application and/or removal of the biodegradable anchoring material 34 can utilize masking or other techniques to selectively apply or selectively remove a portion of the biodegradable anchoring material 34.

In some embodiments, the biodegradable anchoring material 34 can be directly coupled to (without intervening elements) to the elongated tubular member 12. For instance, the biodegradable anchoring material 34 can be manifested as a biodegradable lattice or spun biodegradable matrix that is directly coupled to the elongated tubular member 12 (e.g., to one or both of the first end region 20 and the second end region 22). However, in some embodiments the biodegradable anchoring material 34 can be indirectly coupled to the elongated tubular member 12. For instance, a portion or all of the elongated tubular member 12 can coated, for instance, with a polymer coating as described herein. For example, the elongated tubular member 12 can be a coated elongated tubular member (e.g., coated with silicone or a silicone-based coating) and the biodegradable anchoring material 34 can be indirectly coupled via the polymeric coating to an exterior (e.g., a radial-most surface) of the elongated tubular member 12.

In some embodiments, the biodegradable anchoring material 34, can form a portion of an exterior surface of the stents herein. For instance, the biodegradable anchoring material 34 can form at least a portion of an exterior surface of the first end region 20 and/or the second end region 22 of the stents herein.

In some embodiments, the biodegradable anchoring material 34 can have a uniform profile such as a uniform thickness (e.g., a spun biodegradable matrix with a uniform thickness) and/or uniform configuration (e.g., including uniform sized interstices formed in a biodegradable lattice or between adjacent portions of a spun biodegradable matrix that is disposed on one or more filaments of the elongated tubular member 12). Employing a uniform profile of the biodegradable anchoring material 34 can promote aspects herein such as promoting uniform biodegradation of the entire biodegradable anchoring material 34 (e.g., for a predetermined indwell time period).

However, in some embodiments the profile of the biodegradable anchoring material 34 can be non-uniform. For instance, the biodegradable anchoring material 34 can overlay an entirety of the elongated tubular member 12 but may have a different thickness (radially) and/or have a different configuration (e.g., employ a denser configuration of material with smaller interstices in a portion of a biodegradable lattice) at one of the first end region 20 and the second end region 22 than a profile of the biodegradable anchoring material 34 taken elsewhere (e.g., at the other of the first end region 20 or the second end region 22). Employing different profiles and/or different configurations of the biodegradable anchoring material at the first end region 20 and/or the second end region 22 can promote aspects herein such promoting tissue ingrowth and/or biodegradation at diffing degrees for different portions of the stent 10 thereby permitting the stent 10 to be readily removed (e.g., after a predetermined amount of indwell time).

The biodegradable anchoring material 34 can be formed of a different material than other portions of the stent 10 such as a material that forms the main stent body (e.g., the elongated tubular member 12). For example, the biodegradable anchoring material 34 can be formed of a different material than the first end region 20 and the second end region 22 of the stent 10. In some embodiments, the entire elongated tubular member 12 or at least a portion of (e.g., the portion between the flared regions 28, 30 and/or the portion between the flared regions 24, 26) the intermediate region 18 is formed of a non-biodegradable material (e.g., one or more non-biodegradable filaments) that is a different material than a material (e.g., one or more biodegradable filaments or a spun biodegradable matrix) that forms the biodegradable anchoring material 34. Having the entire elongate tubular member 12 or at least the intermediate region 18 be formed of a non-biodegradable filament and having the biodegradable anchoring material 34 (e.g., which is coupled to or overlays the first end region 20 and the second end region 22) be formed of a different material can promote aspects herein such as ensuring that the stent 10 retains various desired mechanical properties e.g., the ability to readily contract and expand (e.g., contract during delivery and radially expand once delivered to a target site in a body lumen), unlike other approaches such as those that form an entire stent or at least the intermediate region of the stent from a biodegradable material (e.g., a biodegradable material that is covered with a non-biodegradable covering). In some embodiments, the elongated tubular member 12 can be a braided elongate tubular member, for instance, where some or all of the braided elongated tubular member is formed of a non-biodegradable material.

As mentioned, the biodegradable anchoring material 34 can be configured to degrade over time, allowing for removal of the biodegradable biliary stent 10 after a predetermined indwell time. For instance, the biodegradable anchoring material 34 can comprises least one of a magnesium-based alloy, poly-L-lactic acid (PLLA), poly lactic acid (PLA), polyglycolic acid (PGA), polyglycolide-co-L-lactide acid (PGLA), polydioxanone (PDO), or polyglycolide-co-caprolactone (PGCL). In some embodiments, the biodegradable anchoring material 34 comprises one or more of a magnesium-based alloy, PLA, and PGA. In some embodiments, the biodegradable anchoring material 34 comprises a magnesium-based alloy. Examples of suitable magnesium-based alloys which are biodegradable include AZ31, AZ91, ZE41, WE43, ZM21, Mg—Zn—Ca, and Mg—Zn—Ca—Sr, among other magnesium containing alloys that are biodegradable. In some embodiments, the biodegradable anchoring material 34 comprises PLA such as spun PLA. In some embodiments, the biodegradable anchoring material 34 comprises PGA such as spun PGA.

In some embodiments, the biodegradable biliary stents can include an adhesive material (not illustrated). For instance, the adhesive material can be coupled to or otherwise attached to the biodegradable anchoring material 34 through at least one of physical ingress into interstices of the biodegradable anchoring material 34 and chemical reaction. Examples of suitable adhesive materials include various biocompatible and/or biodegradable adhesives such as chitosan and transglutaminase. In some embodiments, the adhesive material can comprise at least one of chitosan and transglutaminase. In some embodiments the adhesive material can be applied to an entire surface (e.g., an exterior surface of the biodegradable anchoring material 34) via spraying, dip coating, or other method of application. The presence of the adhesive material can aid in initial retention of the biodegradable biliary stents herein at a site within a lumen of a patient, can promote longer indwell times, and/or can promote an indwell time to be tailored to a predetermined indwell time, etc. In some embodiments, the adhesive material can be applied to a spun biodegradable matrix such as those described herein.

In some embodiments, a functional coating or solution can be applied to an exterior surface of the elongated tubular member 12, a coating (if present) on the elongated tubular member 12, and/or a biodegradable anchoring material 34. Examples of functional coatings or solutions include polyethylene glycol (PEG) or PEG based coatings or solutions. The functional coating or solution may be configured to alter a surface characteristic of the elongated tubular member or other surface to which the functional coating or solution is applied. For instance, the PEG or PEG based coating may improve adhesion (e.g., increase a degree of hydrophilicity of a surface) between a coating (e.g., a silicone coating) on the elongated tubular member 12 and the biodegradable anchoring material 34 (e.g., a spun biodegradable matrix) applied thereto. For instance, a spun biodegradable matrix can be a functionalized spun biodegradable matrix (e.g., functionalized with a PEG or a PEG derivative) and the adhesive material can be applied to the functionalized spun biodegradable matrix to form an adhesion-enhanced biodegradable anchoring coating. That is, employing a functionalized spun biodegradable matrix can promote application and/or retention of the adhesive material to the spun biodegradable matrix. Similarly, in some embodiments, the adhesive material can be applied to a biodegradable lattice (e.g., forming a functionalized biodegradable lattice).

The biodegradable anchoring material 34 (e.g., represented generally as horizontal elements 34 in FIG. 1) can be manifested as a biodegradable lattice and/or a spun biodegradable matrix. In some embodiments, the biodegradable anchoring material 34 is a biodegradable lattice. The biodegradable lattice may be made of a flexible, bendable material. In some instances, the biodegradable lattice may be formed from suture material, or other biocompatible material. For instance, the biodegradable lattice may be formed of one or more biocompatible and biodegradable filaments which can be woven or otherwise joined together.

The biodegradable lattice may be formed separately from other portions of the stent 10. For example, the biodegradable lattice may be formed separately from the elongated tubular member 12 and may subsequently be coupled or otherwise affixed to the elongated tubular member 12 (e.g., coupled to a first end region 20 and/or the second end region 22). In such embodiments, the biodegradable lattice 34 may be knotted, wound, wrapped, adhesively secured, or otherwise secured to a portion of the elongated tubular member 12. The one or more filaments forming the biodegradable lattices can be woven or braided together in various manners, for instance, to form a plurality of interstices within the biodegradable lattice. The presence of the interstices can promote tissue ingrowth and/or promote various mechanical properties (e.g., flexibility) of the biodegradable lattice. It is contemplated that the biodegradable lattice may be formed from a biodegradable material that may “fall away” from the elongated tubular member 12 or be absorbed by the patient's body after a period of time, such as two to three weeks after a stent is placed.

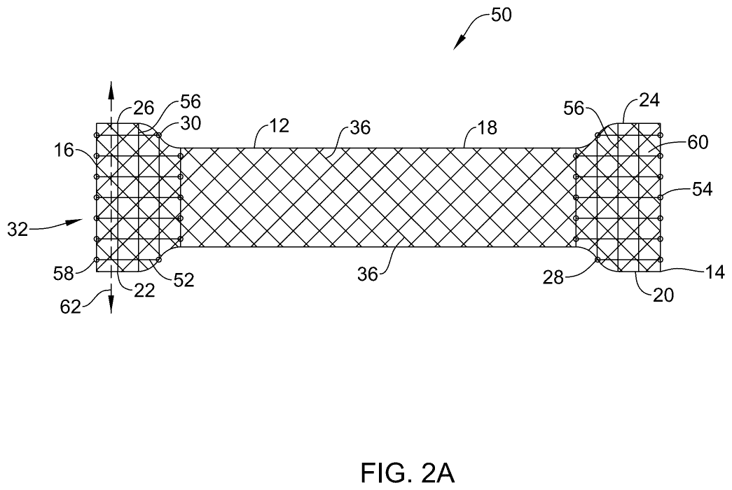

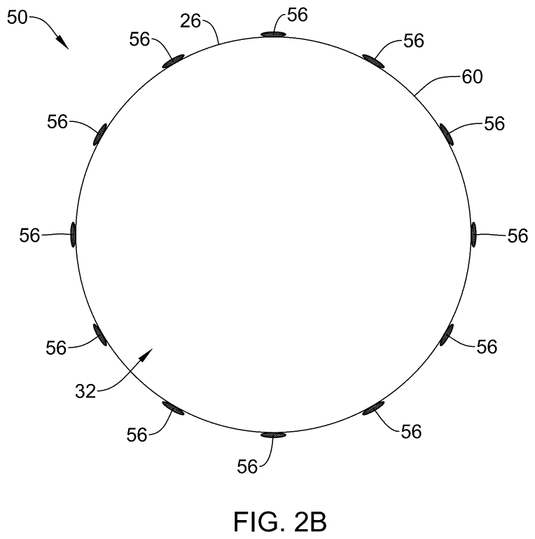

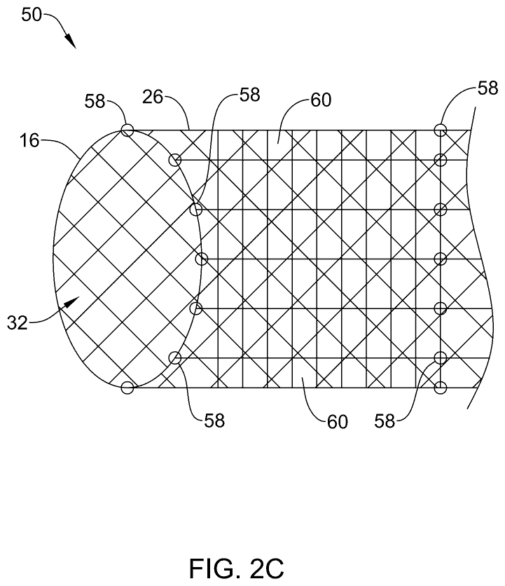

As mentioned, in some embodiments the elongated tubular member 12 can be a braided elongate tubular member. In such embodiments, the biodegradable lattice can be braided or woven into a portion of the braided elongated tubular member 12 such as the first end region 20, the second end region 22, or both the first end region 20 and the second end region 22. FIG. 2A illustrates an example of a biodegradable stent 50, referred to herein as a biodegradable biliary stent 50 (e.g., a braided biodegradable biliary stent), although the stent may be configured for another anatomical location, if desired. The biodegradable biliary stent 50 includes a biodegradable anchoring material in the form of a biodegradable lattice. The biodegradable biliary stent 50 is analogous to the biodegradable stent 10 of FIG. 1, with the change the biodegradable anchoring material (represented generally as element 34 in FIG. 1) is manifested as a biodegradable lattice. As illustrated in FIG. 2A, the biodegradable lattice can be manifested as two individual and separate biodegradable lattices 52, 54, including one or more filaments 56 which are braided or woven to the first end region 20 and the second end region 22, respectively, of the elongated tubular member. At least a proximal end and/or a distal end of the biodegradable lattices 52, 54 can be coupled to the elongated tubular member 12 at one or more junction points (e.g., as represented by elements 58 in FIG. 2A).

For example, an end (e.g., a proximal end) of the biodegradable lattice 52 can be coupled to the second end 16 of the elongated tubular member 12 and an end (e.g., a distal end of the biodegradable lattice 54 can be coupled to the first end 14 of the elongated tubular member 12. As an example, an end (e.g., one or more filaments) of a biodegradable lattice may be looped around a filament of the stent to secure the biodegradable lattice(s) to the stent 10. For instance, as illustrated in FIG. 2A, each of the respective proximal ends and distal ends of the biodegradable lattices 52, 54 can be coupled to the respective proximal end region 20 and the distal end region 22 of the stent 50. Alternatively or in addition, one or more portions of the biodegradable lattices 52, 54 that are located between the distal and proximal ends of the biodegradable lattices 52, 54 can be coupled to (e.g., braided or interwoven with one or more filaments forming the elongated tubular member 12) at one or more alternate or additional junction points located between the proximal and distal ends of the biodegradable lattices 52, 54. In some embodiments, an adhesive or other component (e.g., one or more additional filaments or ties) can be present to couple or aid in coupling the biodegradable lattices 52, 54 to the elongated tubular member 12.

The biodegradable lattices 52, 54 can be the same shape and size, as illustrated in FIG. 2A. However, in some embodiments the biodegradable lattices 52, 54 can be different shapes and/or sizes. For instance, one of the biodegradable lattices 52, 54 can have a thicker profile (radially) than the other of the biodegradable lattices 52, 54. For example, one of the biodegradable lattices 52, 54 that is subjected to or anticipated to be subjected to increased force (e.g., due to peristalsis or higher magnitudes of peristalsis, etc.) can have thicker profile than the other of the biodegradable lattices 52, 54, for instance to promote retention of the biodegradable biliary stent 50 at a target site in a lumen (e.g., in the vasculature) of a patient.

FIG. 2B illustrates a section view (taken along section line 62) of the biodegradable biliary stent 50 of FIG. 2A, while FIG. 2C illustrates a view of an end portion of the biodegradable biliary stent 50 of FIG. 2A. As illustrated in FIGS. 2B and 2C, the one or more filaments 56 forming the biodegradable lattices can be located substantially on an exterior surface of the biodegradable biliary stent 50. For instance, the distal and proximal ends of the biodegradable lattices can be coupled to the distal and/or proximal end regions of the elongated tubular member at one or more junction points and the remaining portions of the one or more filaments of the biodegradable lattices (located between the proximal and distal ends) can extend continuously along an outer surface of the elongated tubular member between the junction points. However, as mentioned in some embodiments the portions of the biodegradable lattices located between the junction points can be interwoven or braided (e.g., in an over under manner, etc.) with the one or more filaments of the elongated tubular member that are located between the junction points. In any case, at least a portion of the biodegradable lattices can form an exterior surface of a portion of the biodegradable biliary stents herein.

As illustrated in FIGS. 2B and 2C, the one or more filaments forming the biodegradable lattices can be spaced apart about a circumference of a portion of the elongated tubular member. Thus, the one or more filaments 56 forming the biodegradable lattices can have interstices 60 located therebetween. For instance, the one or more filaments 56 forming the biodegradable lattices can be the same size, the same shape, and can be spaced equidistantly apart. Having the one or more filaments 56 forming the biodegradable lattices be the same size, the same shape, and can be spaced equidistantly apart can promote aspects herein such a forming uniform interstices within the biodegradable lattices and thereby promoting uniform tissue ingrowth and/or biodegradation of the biodegradable lattices. However, the size, shape, and/or the configuration of the one or more filaments 56 (and thus the interstices 60 therebetween) can be varied.

In some embodiments, the one or more filaments forming the biodegradable lattices can be configured at a non-zero angle to the one or more filaments forming the elongated tubular member 12, for instance, as illustrated in FIGS. 2A-2C. For instance, as illustrated in FIG. 2B each of the biodegradable lattices 52, 54 can be configured at the same relative angle to the one or more filaments forming the respective portion of the elongated tubular member 12. Such a configuration may promote aspects herein such as promoting the same or similar biodegradation rates of the biodegradable lattices 52, 54 and/or promoting uniform or consistent mechanical properties of the biodegradable biliary stent.

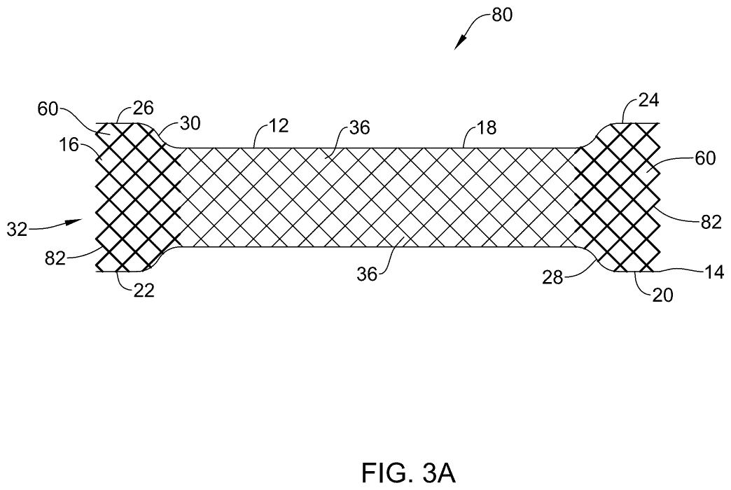

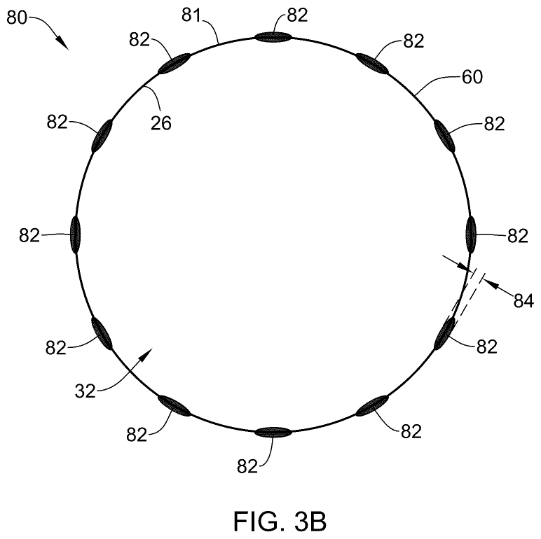

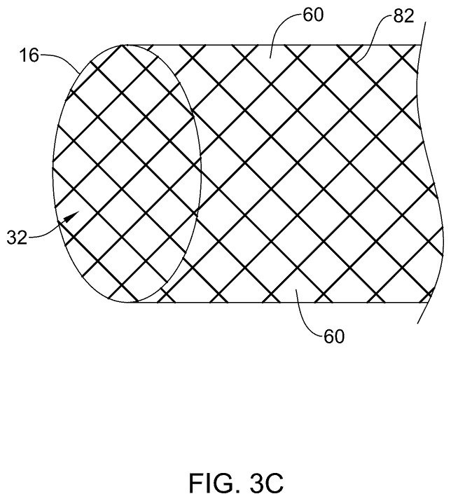

FIG. 3A illustrates an example of a biodegradable stent 80, referred to herein as a biodegradable biliary stent 80, although the stent may be configured for another anatomical location, if desired. The biodegradable biliary stent 80 includes a biodegradable anchoring material in the form of a spun biodegradable matrix 82. That is, the biodegradable biliary stent 80 is analogous to the biodegradable biliary stent 50 of FIG. 1, with the change that the biodegradable anchoring material is manifested as the spun biodegradable matrix 82 rather than the biodegradable lattice of FIG. 2A.

The spun biodegradable matrix 82 can be located at least one of the proximal end region 20 and the distal end region 22. For instance, as illustrated in FIG. 2A the spun biodegradable matrix 82 can be located on each of the proximal end region 20 and the distal end region 22 of the biodegradable biliary stent 50. In some embodiments, the spun biodegradable matrix 82 can be located on each of the proximal end region 20 and the distal end region 22 of the biodegradable biliary stent 80, but not is not located on the intermediate region 18, as illustrated in FIG. 2A. For instance, the spun biodegradable matrix 82 can be located substantially on the one or more filaments that form a portion of the elongated tubular member 12 but does not substantially occupy interstices between the one or more filaments forming the elongated tubular member 12. For example, the spun biodegradable matrix 82 can be located substantially on the one or more filaments or portions thereof that form the proximal end region 20 and the distal end region 22 of the biodegradable biliary stent 80. In some embodiments, a plurality of interstices 60 are present within (between adjacent coated filaments) different portions of the spun biodegradable matrix 82. As mentioned, the presence of the interstices 60 (and in some instances the interstices between the one or more filaments of the elongated tubular member 12) can promote aspects herein such as promoting tissue ingrowth and/or promoting desired mechanical performance (e.g., radial contraction and expansion of the stent 80), for instance as compared to continuous coatings or layers formed by other methodologies.

FIG. 3B illustrates a section view (taken along section line 76 as illustrated in FIG. 3A) of the biodegradable biliary stent 80 of FIG. 3A, while FIG. 3C illustrates a view of an end portion of the biodegradable biliary stent 80 of FIG. 3A. As illustrated in FIGS. 3B and 3C, the spun biodegradable matrix can be located substantially on an exterior surface of the biodegradable biliary stent 80. The biodegradable anchoring material can be formed (e.g., formed only of) a spun biodegradable matrix. For instance, the spun biodegradable matrix can be an electrospun biodegradable matrix (e.g., a traditional electrospun biodegradable matrix and/or a coaxial electrospun biodegradable matrix), an airspun biodegradable matrix, a meltspun biodegradable matrix, a centrifugal spun biodegradable matrix, and/or a biodegradable matrix formed via another type of spinning process. In some embodiments, the spun biodegradable matrix can be an electrospun biodegradable matrix. In some embodiments, the spun biodegradable matrix 82 can be extend circumferentially about one or more filaments. That is, the spun biodegradable matrix 82 can extend circumferentially about an entire exterior surface of one or more filaments (e.g., one or more coated filaments) forming the elongated tubular member 12. For instance, as illustrated in FIG. 2B the spun biodegradable matrix 82 being located circumferentially about the one or more filaments that form the proximal end region 26 of the elongated tubular member 12.

A thickness 84 (in a radial direction) of the biodegradable anchoring material (e.g., a spun biodegradable matrix 82) can be in a range from about 100 nanometers (nm) to about 700 nm, from about 200 nm to about 700 nm, from about 300 nanometers to about 600 nm, or from about 450 nm to about 700 nm. As mentioned, the thickness of the biodegradable anchoring material (e.g., a spun biodegradable matrix) can be uniform or can vary (e.g., be non-uniform) along a length (longitudinally) of the biodegradable anchoring material. For instance, a portion of the spun biodegradable matrix 52 can have a thicker profile (radially) than another portion spun biodegradable matrix 52. For example, a portion of the spun biodegradable matrix 52 that is subjected to or anticipated to be subjected to increased force (e.g., due to peristalsis or higher magnitudes of peristalsis, etc.) can have thicker profile than another portion of the spun biodegradable matrix 52, for instance, to promote retention of the biodegradable biliary stent at a target site in a body lumen (e.g., in the vasculature of a patient). For instance, a portion of the spun biodegradable matrix 82 located at the proximal end region 20 can have a different thickness than a portion of the spun biodegradable matrix located at the distal end region and/or a thickness of the spun biodegradable matrix can vary (longitudinally) along one of both of the proximal end region 20 and the distal end region 22.

As mentioned, in some embodiments the biodegradable biliary stents herein can be coated biodegradable biliary stents (e.g., fully coated biodegradable biliary stents) that are coated with silicone or another polymeric material. For instance, the biodegradable biliary stent 80 can be a fully coated stent including a coating (e.g., a silicone coating) overlaying an entire elongated tubular member 12 of the stent. As illustrated in FIG. 3B, the stent 80 can include a coating 81 that extends circumferentially about the entire exterior surface of the proximal end region 26. In such embodiments, the spun biodegradable matrix 82 can be located partially within the coating 81 and/or can be located on an exterior surface of the coating 81. For example, the coating 81 may be a cross-linked coating (e.g., a silicone cross-linked coating) where the spun biodegradable matrix 82 is applied during and/or subsequent to cross-linking of the coating 81. For instance, in some embodiments the coating is a silicone coating and the spun biodegradable matrix 82 can be an electrospun biodegradable layer that is applied via electrospinning prior to or at least during cross-linking of the silicone coating. Application to the coating 81 prior to or at least during cross-linking of the silicone coating can promote aspects herein, such as promoting application of the spun biodegradable coating substantially to the one or more filaments forming a portion (e.g., the proximal end region 26 and/or the distal end region 24) of the elongated tubular member 12, as compared to other approaches that may apply a biodegradable layer or coating to a coated stent (e.g., which may therefore not have interstices 60 formed within the spun biodegradable matrix 82).

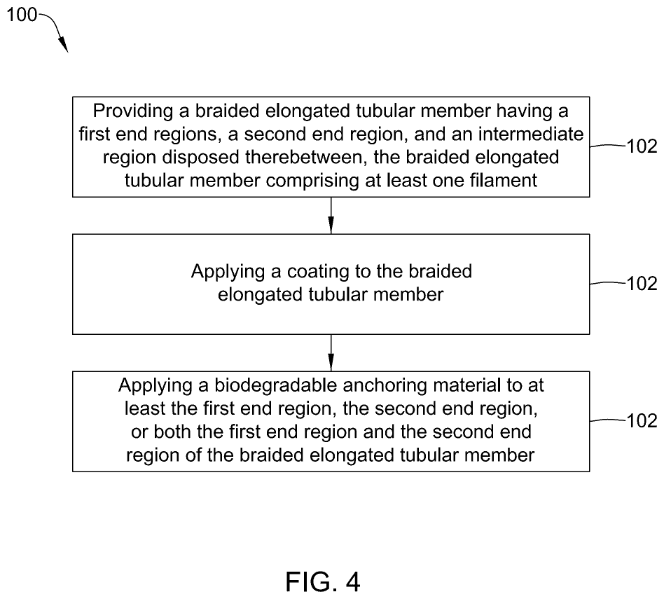

FIG. 4 is an illustrative method 100 of forming a coated biodegradable stent. At 102, the method 100 includes providing a braided elongated tubular member having a first end region, a second end region, and an intermediate region disposed therebetween, the braided elongated tubular member comprising at least one filament. Examples of suitable braided elongated tubular members are described herein. At 104, the method 100 includes applying a coating to the braided elongated tubular member. As mentioned, the coating can be a silicone coating or other coating such as another type of cross-linked coating. For instance, the coating can be a silicone coating that is applied to an entire braided elongated tubular member (e.g., the braided elongated tubular member can be a fully coated braided elongated tubular member).

At 106, the method 100 includes applying a biodegradable anchoring material to at least the first end region, the second end region, or both the first end region and the second end region of the braided elongated tubular member. As mentioned, the biodegradable anchoring material can be a biodegradable lattice or a spun biodegradable matrix. The biodegradable anchoring material can be applied to the braided elongated tubular member as described herein. For instance, a biodegradable lattice can be woven or braided onto the braided elongated tubular member at one or more junction points. For example, applying the biodegradable lattice comprises braiding or weaving the biodegradable lattice onto the first end region, the second end region, or both the first end region and the second end region via one or more junction points, as described herein. In some embodiments, the biodegradable anchoring material can be a spun biodegradable matrix that is spun (e.g., electrospun, etc.) onto one or more filaments forming the braided elongated tubular member, as described herein. While the method 100 pertains to a coated biliary stent, in some embodiments the methods herein can be employed with other types of stents and/or with uncoated biliary stents (e.g., with an absence of applying a coating to the braided elongated tubular member).

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304 V, 304 L, and 316 LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially”linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (°C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. In some other embodiments, a superelastic alloy, for example a superelastic nitinol can be used to achieve desired properties.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

In some embodiments, a degree of Magnetic Resonance Imaging (MRI) compatibility is imparted into the stents or delivery systems. For example, the stents or delivery systems or portions thereof, may be made of a material that does not substantially distort the image and create substantial artifacts (i.e., gaps in the image). Certain ferromagnetic materials, for example, may not be suitable because they may create artifacts in an MRI image. The stents or delivery systems or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in various details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A biodegradable stent comprising:
an elongated tubular member having a first end region, a second end region, and an intermediate region disposed therebetween, the elongated tubular member comprising at least one filament; and
a biodegradable anchoring material that is coupled to and overlays a portion of the elongated tubular member, wherein the biodegradable anchoring material is coupled to and overlays at least to the first end region, the second end region, or both the first end region and the second end region.

2. The biodegradable stent of claim 1, wherein the biodegradable anchoring material is coupled to and overlays both the first end region and the second end region of the elongated tubular member.

3. The biodegradable stent of claim 1, wherein the biodegradable anchoring material is only coupled to and overlays the first end region, the second end region, or both the first end region and the second end region.

4. The biodegradable stent of claim 1, wherein the biodegradable anchoring material is configured to degrade over time, allowing for removal of the biodegradable biliary stent after a predetermined indwell time.

5. The biodegradable stent of claim 1, wherein the biodegradable anchoring material comprises least one of a magnesium-based alloy, one of poly-L-lactic acid (PLLA), polylactic acid (PLA), polyglycolic acid (PGA), polyglycolide-co-L-lactide acid (PGLA), polydioxanone (PDO), or polyglycolide-co-caprolactone (PGCL).

6. The biodegradable stent of claim 1, biodegradable anchoring material defines a plurality of interstices.

7. The biodegradable stent of claim 1, wherein the biodegradable anchoring material is a biodegradable lattice.

8. The biodegradable stent of claim 7, wherein the elongated tubular member is a braided elongate tubular member, and wherein the biodegradable lattice is braided or woven into the first end region, the second end region, or both the first end region and the second end region.

9. The biodegradable stent of claim 1, wherein the biodegradable anchoring material is a spun biodegradable matrix.

10. The biodegradable stent of claim 9, wherein the spun biodegradable matrix further comprises an electrospun biodegradable matrix.

11. The biodegradable stent of claim 1, wherein the biodegradable anchoring material includes an adhesive material.

12. The biodegradable stent of claim 11, wherein the adhesive material comprises at least one of chitosan and transglutaminase.

13. The biodegradable stent of claim 11, wherein the biodegradable anchoring material is functionalized, and wherein adhesive material is coupled to the functionalized biodegradable anchoring material.

14. The biodegradable stent of claim 1, wherein the elongated tubular member is fully covered.

15. The biodegradable stent of claim 1, wherein the elongated tubular member is a braided elongated tubular member, wherein the first end region is a flared first end region, and wherein the second end region is a flared second end region.

16. A coated biodegradable stent comprising:
a braided elongated tubular member having a first end region, a second end region, and an intermediate region disposed therebetween, the elongated tubular member comprising at least one filament and a coating overlaying at least a portion of the elongated tubular member, wherein the at least on filament is a non-biodegradable filament; and
a biodegradable anchoring material coupled to a portion of the braided elongated tubular member, wherein the biodegradable anchoring material is coupled to at least to the first end, the second end, or both the first end region and the second end region of the braided elongated tubular member, wherein the biodegradable anchoring material comprises at least one of a magnesium-based alloy, polylactic acid (PLA), and polyglycolic acid (PGA) and is configured to provide anchoring and biodegrade over time.

17. A method of manufacturing a coated biodegradable stent, comprising:
providing a braided elongated tubular member having a first end region, a second end region, and an intermediate region disposed therebetween, the braided elongated tubular member comprising at least one filament;
applying a coating to the braided elongated tubular member; and
applying a biodegradable anchoring material to at least the first end region, the second end region, or both the first end region and the second end region of the braided elongated tubular member.

18. The method of claim 17, wherein biodegradable anchoring material further comprises a biodegradable lattice, and wherein applying the biodegradable lattice comprises braiding or weaving the biodegradable lattice onto the first end region, the second end region, or both the first end region and the second end region.

19. The method of claim 17, wherein biodegradable anchoring material further comprises a biodegradable anchoring coating, and wherein applying the biodegradable coating comprises electrospinning a biodegradable polymer onto at least the first end region, the second end region, or both the first end region and the second end region of the elongated tubular member while applying or cross-linking the coating to the braided elongated tubular member.

20. The method of claim 19, further comprising forming an adhesion-enhanced biodegradable anchoring coating by:
functionalizing the biodegradable anchoring coating by applying polyethylene glycol (PEG) or a PEG derivative to the biodegradable anchoring coating; and applying an adhesive material to the functionalized biodegradable coating to form the adhesion-enhanced biodegradable anchoring coating.
